(19) United States
(12) Patent Application Publication
(10) Pub. No.: US 2025/0352781 A1
(43) Pub. Date: Nov. 20, 2025

(54) DELIVERY APPARATUS, SYSTEMS, AND METHODS FOR MEDICAL DEVICES

(71) Applicant: STAR BP, INC., SPRING, TX (US)

(72) Inventors: J. Ryan STANFIELD, Sandy, UT (US); Michael A. VLADOVICH, Edmond, OK (US)

(21) Appl. No.: 19/201,570

(22) Filed: May 7, 2025

Related U.S. Application Data
(60) Provisional application No. 63/649,028, filed on May 17, 2024.

(51) Int. Cl. A61M 60/865; A61M 60/122

(52) U.S. Cl. CPC A61M 60/865; A61M 60/122; A61M 2205/04; A61M 2210/125

(57) ABSTRACT

Systems and methods assist with delivery of a medical device, such as a cardiac assistance device, within a patient. The system may include a sleeve or sheath for temporary covering of external ports into the cardiac assistance device during a procedure for the delivery. A pusher rod may also facilitate the procedure for the delivery. The cardiac assistance device may further contain grooves that may be engaged with a lasso for pulling the device through the vasculature system to position the device, to attach the device to an anchor, or to remove the device from the anchor.

### CROSS-REFERENCE TO RELATED APPLICATIONS

This application is a nonprovisional application which claims priority from U.S. provisional application No. 63/649,028, filed May 17, 2024, which is incorporated by reference herein in its entirety.

### FIELD

Apparatus, systems, and methods enable implantation of medical devices, such as for facilitating blood flow.

### BACKGROUND

Implantation of non-surgical implantable medical devices may require insertion and manipulation of the device through one or more incisions in a patient. Examples of such devices include cardiac assistance devices, which may assist in pumping blood through the vasculature system of the patient. The delivery of the device thus may require navigating through veins/arteries to position the device and perhaps engage the device with some form of dock. Such inherently difficult tasks often cannot be achieved consistently and successfully with prior approaches and are complicated by various factors. For example, external surface features of the device necessary for its functioning once deployed may interfere with or create problems during the delivery and/or docking of the device.

### SUMMARY

For one embodiment, a system for performing a medical delivery procedure includes a cardiac assistance device for facilitating blood flow from a first anatomical space of a patient to a second anatomical space of the patient. The system further includes an anchor device arrangeable across the first and second anatomical spaces and a cover arranged about the cardiac assistance device. The cover is configured to cover a blood flow port of the cardiac assistance device during the medical delivery procedure and to be releasable from about the cardiac assistance device for retrieval of the cover after the cardiac assistance device is engaged in the anchor device.

In one embodiment, a cardiac assistance device for delivery into a patient includes an outlet for exiting flow of blood at a transverse end face of a first end of the cardiac assistance device and inlet ports disposed around a circumference on a second end of the cardiac assistance device for inflow of the blood into the cardiac assistance device. The second end accommodates for thickness of a cover over the inlet ports by having a relatively smaller diameter than the first end of the cardiac assistance device. The cardiac assistance device further includes an engaging profile disposed around a circumference of the cardiac assistance device and located on the first end of the cardiac assistance device for mating with a corresponding receiving profile of an anchor device in the patient. In addition, the cardiac assistance device includes a circumferential groove located on the first end of the cardiac assistance device between the transverse end face and the engaging profile for retaining a lasso to facilitate pulling the cardiac assistance device in the patient.

According to one embodiment, a method of delivering a cardiac assistance device through a vasculature system includes implanting an anchor device across first and second anatomical spaces of a patient and moving the cardiac assistance device through the vasculature system of the patient toward the anchor device while a cover is arranged about the cardiac assistance device to shield a blood flow port of the cardiac assistance device. The method further includes engaging the cardiac assistance device with the anchor device and releasing the cover from the cardiac assistance device. Further, the method includes retrieving the cover from the patient after the cardiac assistance device is engaged in the anchor device.

### BRIEF DESCRIPTION OF THE DRAWINGS

The present disclosure is best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is an illustration of an anchor arranged to provide a bore across two anatomical spaces, in accordance with various aspects of the present disclosure.

FIG. 2 is an illustration of a cardiac assistance device and sleeve (shown transparent) for placement within the bore of the anchor in FIG. 1, in accordance with various aspects of the present disclosure.

FIG. 3 is an illustration of the sleeve, in accordance with various aspects of the present disclosure.

FIG. 4 is an illustration of the cardiac assistance device with a sheath and pusher, in accordance with various aspects of the present disclosure.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Methods and systems relate to delivery and implantation of medical devices, such as a cardiac assistance device or other medical devices that are not easily collapsible or devices implanted into locations that are not easily accessible or traversable. Some embodiments provide for the cardiac assistance devices, systems, and methods for delivery thereof in a patient. More specifically, cardiac assistance devices and methods may relate to a device or pump that facilitates directional blood flow from one anatomical space to a second anatomical space in a patient's heart or vasculature and circulatory system.

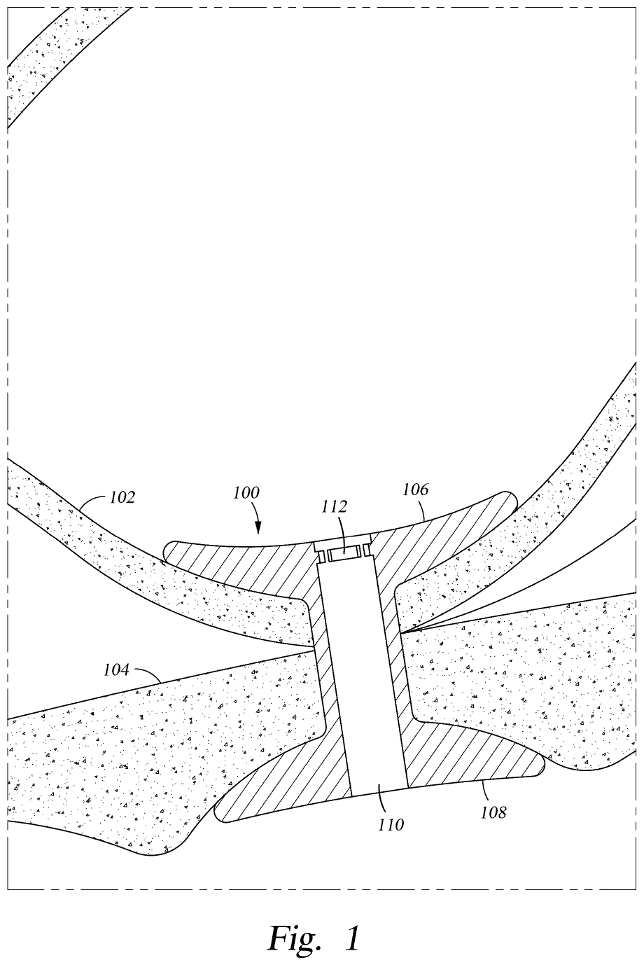

FIG. 1 shows an anchor device 100 arranged across two anatomical spaces defined by first and second tissues 102, 104. In some embodiments, the anchor device 100 includes a first flange 106 adjacent where the anchor device 100 traverses through the first tissue 102 and a second flange 108 adjacent where the anchor device 100 traverses through the second tissue 104. The first and second flanges 106, 108 may be configured to respectively engage the first and second tissues 102, 104 in a fluid tight configuration to seal the anchor device 100 within the first and second tissues 102, 104. The anchor device 100 may be a stent, graft, stent graft component, or other conduit.

As shown in FIG. 1, the anchor device 100 creates a fluidic connection via a bore 110 through the anchor device 100 between the anatomical spaces, such as the aorta and an atrium or ventricle. One example of the two anatomical spaces is the left atrium and aorta. The anchor device 100 may also be used to create a fluidic connection between spaces, tissue structures, conduits, organs, and the like, and combinations thereof.

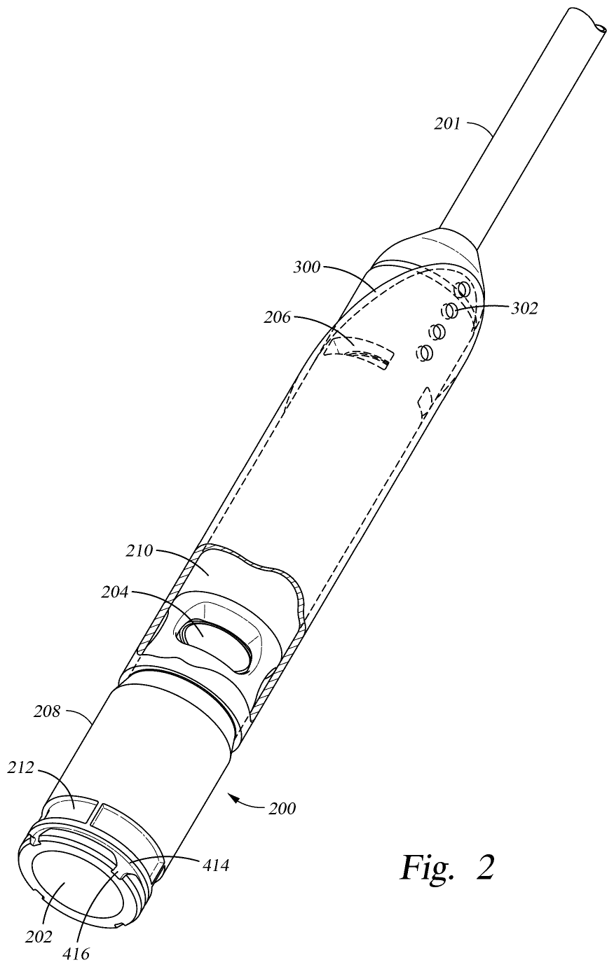

The bore 110 through the anchor device 100 includes a receiving profile 112 for mating with an engaging profile 212 (such as shown in FIG. 2) on an outside of a medical device, which may be a cardiac assistance device, to be positioned in the anchor device 100. In alternative embodiments, the anchor device 100 can be a seat for an occlusive element, for example, a balloon. The occlusive element can shunt between the left atrium and aorta prior to pump deployment. The receiving profile 112 and the engaging profile 212 define complimentary shapes to one another for locking the medical device into the anchor device 100. For example, the receiving profile 112 may be protrusions, such as rectangular islands spaced around an inner circumference of the bore 110, that match with corresponding shaped and spaced recesses, such as the engaging profile 212, into an outer circumference of the medical device. Alternatively, the receiving profile 112 may include recesses into the bore 110 that receive mating protrusions extending outward on the medical device.

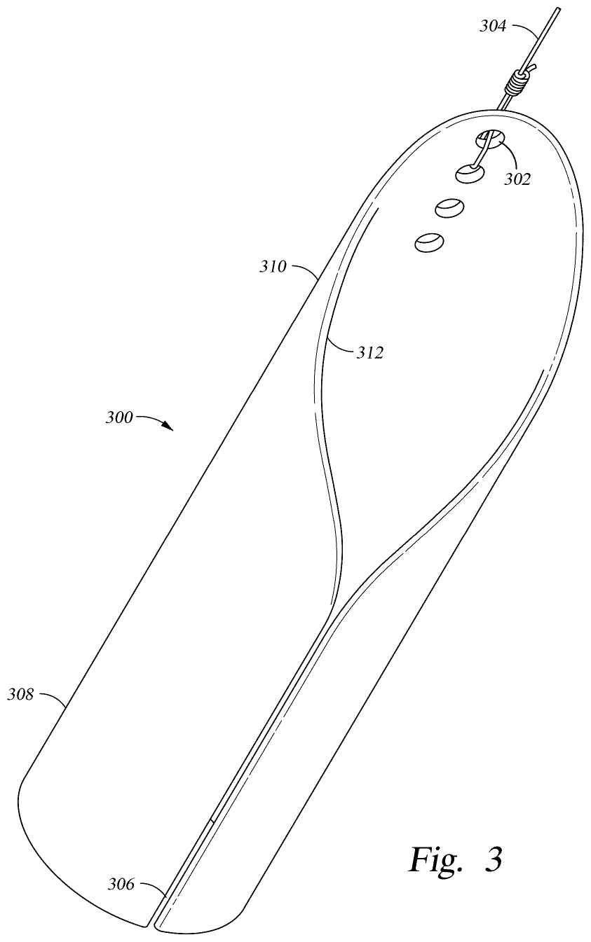

FIG. 2 depicts a cardiac assistance device 200 and a sleeve 300 forming a temporary cover that as shown in FIG. 3 is split or circumferentially non-continuous along the entire length of the sleeve 300. The cardiac assistance device 200 may be configured for facilitating blood flow from a first anatomical space to a second anatomical space or from a first tissue structure to a second tissue structure. One example is directing blood flow from the left atrium of a patient to an aorta of the patient.

The cardiac assistance device 200 may include a cable 201 coupled to an end of the cardiac assistance device 200 opposite of an outlet 202. The cable 201 may provide power/control to the cardiac assistance device 200 for pumping of blood through the cardiac assistance device 200. Blood flow exits the outlet 202 at a transverse end face of the cardiac assistance device 200. Several primary inlet ports 204 and, in some embodiments, secondary inlet ports 206 that are all disposed around a circumference of the cardiac assistance device 200, permit inflow of the blood into the cardiac assistance device 200. For some embodiments, directional flow through the cardiac assistance device 200 may be reversed switching what would be inlets and outlets.

The sleeve 300, in certain instances and as is shown in FIG. 2, may be arranged about the cardiac assistance device 200 and configured to cover some, or all, of the primary inlet ports 204 and/or the secondary inlet ports 206 of the cardiac assistance device 200. The primary and secondary inlet ports 204, 206 being shielded by the sleeve 300 during deployment of the cardiac assistance device 200 helps ensure the receiving profile 112 of the anchor device 100 or any other obstructions do not catch or hang into the primary and secondary inlet ports 204, 206 causing damage to the patient, anchor device 100 or cardiac assistance device 200 or otherwise inhibiting delivery of the cardiac assistance device 200.

The sleeve 300 further allows for an operator to overdrive or over-advance the cardiac assistance device 200. Such overdrive may occur as the outlet 202 of the cardiac assistance device 200 is inserted into the anchor device 100 and the engaging profile 212 of the cardiac assistance device 200 passes across and beyond the receiving profile 112 of the anchor device 100. The sleeve 300 then prevents the receiving profile 112 of the anchor device 100 from coming into potential engaging contact with the primary and secondary inlet ports 204, 206 of the cardiac assistance device 200. The operator can then reposition the cardiac assistance device 200 as desired back to align the engaging and receiving profiles 212, 112 locking the cardiac assistance device 200 into the anchor device 100.

In some embodiments, an end of the sleeve 300 abuts a larger diameter portion 208 of the cardiac assistance device 200 with the sleeve 300 surrounding a reduced diameter portion 210 of the cardiac assistance device 200 where the primary and secondary inlet ports 204, 206 are located. A polymeric material may form the sleeve 300. Since the larger diameter portion 208 has a greater diameter than the reduced diameter portion 210, thickness of the sleeve 300 may match the difference in diameter between the larger diameter portion 208 and the reduced diameter portion 210 for providing an area of constant, or substantially uniform, outer dimension along the sleeve 300 and the larger diameter portion 208 of the cardiac assistance device 200 to further facilitate delivery of the cardiac assistance device 200.

FIG. 3 shows the sleeve 300 with a gap 306 that makes the sleeve 300 circumferentially non-continuous to facilitate initial installation onto the cardiac assistance device 200, securement throughout deployment of the cardiac assistance device 200 and removal from the cardiac assistance device 200. In some embodiments, the sleeve 300 secures to the cardiac assistance device 200 by a close or biased fit around the cardiac assistance device 200 at least in part due to the sleeve 300 being elastically expanded at the gap 306 to clamp around the cardiac assistance device 200. In some embodiments, the sleeve 300 may include a first portion 308 having a substantially cylindrical shape except for the gap 306 and a second portion 310 that transitions along a taper 312 from the first portion 308 as the cylindrical shape is progressively cutaway.

The sleeve 300 further may include one or more apertures 302 illustrated as four radial holes extending through the second portion 310 of the sleeve 300. The apertures 302 may interface with a fiber 304 that is arranged through and tied or coupled with the apertures 302 in the sleeve 300 to secure the fiber 304 to the sleeve 300. The gap 306 may extend longitudinally along the sleeve 300 circumferentially offset opposite (e.g., 180 degrees) relative to the apertures 302.

In operation, the operator delivers the cardiac assistance device 200 with the sleeve 300 attached to the anchor device 100 and must then remove and retrieve the sleeve 300 once the cardiac assistance device 200 is positioned as desired in the anchor device 100. The fiber 304 facilitates removing the sleeve 300 from the cardiac assistance device 200 in response to tension applied to the fiber 304 that may extend out of the patient. The sleeve 300 as shaped by the first and second portions 308, 310 and location of the apertures 302 results in the tension applied by the fiber 304 causing the gap 306 to open for release of the sleeve 300 from about the cardiac assistance device 200. Continued pulling of the fiber 304 may retrieve the sleeve 300 from the patient.

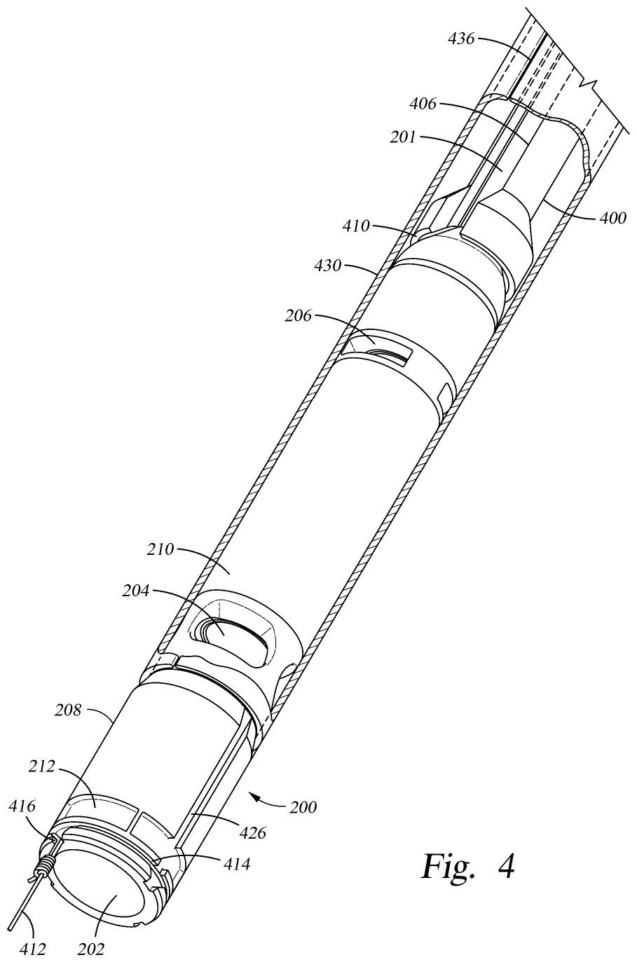

FIG. 4 illustrates the cardiac assistance device 200 with a sheath 430 (as a cover alternative for the sleeve 300 shown in FIG. 2) that is shown as a tube shape disposed covering the primary and secondary inlet ports 204, 206 and may continue over a pusher 400. While depicted by example with the sheath 430, other embodiments may include the pusher 400 used with the sleeve 300, with or without the sheath 430. Like the sleeve 300, the sheath 430 may shield the primary and secondary inlet ports 204, 206 from hanging on obstructions including the receiving profile 112 of the anchor device 100 during delivery of the cardiac assistance device 200. Also, the sheath 430 may create an area of constant, or substantially uniform, outer dimension along the sheath 430 and the larger diameter portion 208 of the cardiac assistance device 200 or the entirety of the cardiac assistance device 200.

During the procedure for delivery of the cardiac assistance device 200, the operator often first runs a wire in the patient to establish a path to be taken to get the cardiac assistance device 200 to the anchor device 100. The wire remains in the patient during the procedure so that the cardiac assistance device 200 follows along the wire. The cardiac assistance device 200 thereby may include a longitudinal channel 426 in the outer surface of the larger diameter portion 208 to act as a guide for passing over the wire. The sheath 430 may be sized to accommodate the wire. For embodiments with the sleeve 300 shown in FIG. 2 and FIG. 3, the sleeve 300 may be oriented with the longitudinal channel 426 aligned with the gap 306 in the sleeve 300 so that the wire may pass along both the longitudinal channel 426 and the gap 306.

The pusher 400 fits around the cable 201 of the cardiac assistance device 200 and may include a longitudinal slot 406 sized for freely placing the cable 201 into or out of the pusher 400 along the longitudinal slot 406. A distal end of the pusher 400 forms a seat 410 for abutting the cardiac assistance device 200 coupled to the cable 201. The pusher 400 around the cable 201 in combination with relative greater stiffness than the cable 201 alone facilitates the operator being able to apply force to the pusher 400 and transfer that force to the cardiac assistance device 200 for delivery of the cardiac assistance device 200 to the anchor device 100.

The sheath 430 may be formed of a polymeric material and include a longitudinal score 436 where part of the material is removed or missing. After delivery of the cardiac assistance device 200 into the anchor device as desired, pulling on the sheath 430 causes splitting of the sheath 430 along the longitudinal score 436 thereby releasing the sheath 430 from around the cardiac assistance device 200. Further pulling on the sheath 430 along with the pusher 400 then retrieves the sheath 430 and the pusher 400 from the patient. In alternative embodiments, the pusher 400 may be retrieved through the sheath 430 after delivering the cardiac assistance device 200 into the anchor device 100 and prior to pulling and retrieving the sheath 430. The longitudinal slot 406 enables removal of the pusher 400 from the cable 201 as the pusher 400 is being retrieved. The sheath 430 also frees of the cable 201 as the sheath 430 is being retrieved due to having been split at the longitudinal score 436.

In some embodiments, the cardiac assistance device 200 as shown in FIG. 2 and FIG. 4 includes a circumferential groove 414 located in the outer surface of the cardiac assistance device 200 between where the cardiac assistance device 200 terminates at the outlet 202 and where the engaging profile 212 are on the outer surface of the cardiac assistance device 200. The circumferential groove 414 interfaces with and retains a lasso 412 (shown in FIG. 4 as a wire with a snare end) introduced into the patient for pulling the cardiac assistance device 200. As known in the delivery procedures, a loop is often established between two veins/arteries to provide access to both an end of the cardiac assistance device 200 with the cable 201 and an opposite end at the outlet 202. In operation, the lasso 412 encircles the cardiac assistance device 200 and rests in the circumferential groove 414 to be out of the way from interfering with the delivery of the cardiac assistance device 200, such as pulling the cardiac assistance device 200 through the anchor device 100 until locked in the anchor device 100 or pulling cardiac assistance device 200 to release from the anchor device 100. The circumferential groove 414 also keeps the lasso 412 from slipping off the cardiac assistance device 200 while pulling. An end of the lasso 412 extending away from the cardiac assistance device 200 extends to out of the patient for pulling by the operator. Part of the end of the lasso 412 passes through a longitudinal groove 416 formed in the outer surface of the cardiac assistance device 200. The longitudinal groove 416 thus passes between the circumferential groove 414 and where the cardiac assistance device 200 terminates at the outlet 202.

Ability to push and/or pull the cardiac assistance device 200 with the pusher 400 and/or the lasso 412 facilitates the procedure for delivery of the cardiac assistance device 200. For example, having an option to either push or pull the cardiac assistance device 200 means an operator is not reliant on, or limited to, only a single type of force applied to the cardiac assistance device 200. In addition, some delivery procedures may benefit from pulling with the lasso 412 instead of pushing the cardiac assistance device 200.

Methods may use the apparatus and systems disclosed above. In one embodiment, a method of delivering the cardiac assistance device 200 through a vasculature system includes implanting the anchor device 100 across first and second anatomical spaces of the patient. The operator then moves the cardiac assistance device 200 through the patient toward the anchor device 100 while the cover (sleeve 300 or sheath 430) is arranged about the cardiac assistance device 200 to shield the primary and secondary inlet ports 204, 206 of the cardiac assistance device 200. The movement of the cardiac assistance device 200 may be facilitated with the pusher 400 and/or the lasso 412. The method further includes engaging the cardiac assistance device 200 with the anchor device 100, releasing the cover (sleeve 300 or sheath 430) from about the cardiac assistance device 200, and retrieving the cover (sleeve 300 or sheath 430) from the patient after the cardiac assistance device 200 is engaged in the anchor device 100.

The foregoing outlines features of several embodiments so that a person of ordinary skill in the art may better understand the aspects of the present disclosure. Such features may be replaced by any one of numerous equivalent alternatives, only some of which are disclosed herein. One of ordinary skill in the art should appreciate that they may readily use the present disclosure as a basis for designing or modifying other processes and structures for carrying out the same purposes and/or achieving the same advantages of the embodiments introduced herein. One of ordinary skill in the art should also realize that such equivalent constructions do not depart from the spirit and scope of the present disclosure and that they may make various changes, substitutions, and alterations herein without departing from the spirit and scope of the present disclosure.

What is claimed is:

1. A system for performing a medical delivery procedure, comprising:
a cardiac assistance device for facilitating blood flow from a first anatomical space of a patient to a second anatomical space of the patient;
an anchor device arrangeable across the first and second anatomical spaces; and
a cover arranged about the cardiac assistance device and configured to cover a blood flow port of the cardiac assistance device during the medical delivery procedure and to be releasable from about the cardiac assistance device for retrieval of the cover after the cardiac assistance device is engaged in the anchor device.

2. The system of claim 1, wherein the cover includes a gap making the cover non-continuous circumferentially along entirety of the cover.

3. The system of claim 2, wherein the cover includes at least one aperture to be leveraged in retrieval of the cover by being disposed in the cover circumferentially opposite of the gap.

4. The system of claim 3, further including a fiber secured to the cover via the aperture in the cover and extending from the cover for pulling of the fiber by an operator to remove the cover from the cardiac assistance device disposed in the patient.

5. The system of claim 1, further comprising a lasso configured to be retained in a circumferential groove disposed in the cardiac assistance device to facilitate pulling of the cardiac assistance device in the patient during the medical delivery procedure.

6. The system of claim 1, further comprising a pusher abutting the cardiac assistance device and disposed partially around a cable extending from the cardiac assistance device to facilitate force transfer to the cardiac assistance device during the medical delivery procedure.

7. The system of claim 1, further comprising:
a lasso configured to be retained in a circumferential groove disposed in the cardiac assistance device to facilitate pulling of the cardiac assistance device in the patient during the medical delivery procedure; and
a pusher abutting the cardiac assistance device and disposed partially around a cable extending from the cardiac assistance device to facilitate force transfer to the cardiac assistance device during the medical delivery procedure.

8. The system of claim 1, wherein a first portion of the cover has a cylindrical shape and a second portion of the cover transitions along a taper from the first portion as the cylindrical shape is progressively cutaway.

9. A cardiac assistance device for delivery into a patient, comprising:
an outlet for exiting flow of blood at a transverse end face of a first end of the cardiac assistance device;
inlet ports disposed around a circumference on a second end of the cardiac assistance device for inflow of the blood into the cardiac assistance device, wherein the second end accommodates for thickness of a cover over the inlet ports by having a relative smaller diameter than the first end of the cardiac assistance device;
an engaging profile disposed around a circumference of the cardiac assistance device for mating with a corresponding receiving profile of an anchor device in the patient, wherein the engaging profile is located on the first end of the cardiac assistance device; and
a circumferential groove for retaining a lasso to facilitate pulling the cardiac assistance device in the patient, wherein the circumferential groove is located on the first end of the cardiac assistance device between the transverse end face and the engaging profile.

10. The cardiac assistance device of claim 9, further comprising the cover disposed over the inlet ports.

11. A method of delivering a cardiac assistance device through a vasculature system, comprising:
implanting an anchor device across first and second anatomical spaces of a patient;
moving the cardiac assistance device through the vasculature system of the patient toward the anchor device while a cover is arranged about the cardiac assistance device to shield a blood flow port of the cardiac assistance device;
engaging the cardiac assistance device with the anchor device;
releasing the cover from about the cardiac assistance device; and
retrieving the cover from the patient after the cardiac assistance device is engaged in the anchor device.

12. The method of claim 11, wherein the releasing of the cover includes applying tension to a fiber secured to the cover.

13. The method of claim 11, wherein the cover includes a gap making the cover non-continuous circumferentially along entirety of the cover.

14. The method of claim 13, wherein the releasing the cover includes applying tension to a fiber secured to the cover via an aperture disposed in the cover circumferentially opposite of the gap.

15. The method of claim 11, wherein the moving of the cardiac assistance device includes pulling the cardiac assistance device from a first end of the cardiac assistance device and pushing the cardiac assistance device from a second end of the cardiac assistance device.

16. The method of claim 11, wherein the moving of the cardiac assistance device includes positioning a lasso in a circumferential groove disposed in the cardiac assistance device and pulling on an end of the lasso outside of the patient.

17. The method of claim 11, wherein the moving of the cardiac assistance device includes transferring force to the cardiac assistance device via a pusher abutting the cardiac assistance device and disposed partially around a cable extending from the cardiac assistance device.

18. The method of claim 11, wherein the moving of the cardiac assistance device includes:
positioning a lasso in a circumferential groove disposed in the cardiac assistance device and pulling on an end of the lasso outside of the patient; and
transferring force to the cardiac assistance device via a pusher abutting the cardiac assistance device and disposed partially around a cable extending from the cardiac assistance device.

19. The method of claim 11, wherein a first portion of the cover has a cylindrical shape and a second portion of the cover transitions along a taper from the first portion as the cylindrical shape is progressively cutaway.

20. The method of claim 11, further comprising retrieving from the patient a pusher used to transfer force during the moving of the cardiac assistance device, wherein the pusher is retrieved through part of the cover that extends beyond the cardiac assistance device.
